(19) United States
(12) Patent Application Publication
(10) Pub. No.: US 2011/0195482 A1
(43) Pub. Date: Aug. 11, 2011

(54) SERINE PROTEASE ISOLATED FROM THE VENOM OF BOMBUS IGNITUS AS FIBRINOGENOLYTIC AND FIBRINOLYTIC ENZYMES

(75) Inventors: Byung Rae Jin, Gyeonggi-Do (KR); Young Moo Choo, Busan (KR); Kwang Sik Lee, Busan (KR); Yeon Ho Je, Gyeonggi-Do (KR); Hyung Joo Yoon, Gyeonggi-Do (KR); Hung Dae Sohn, Busan (KR)

(73) Assignee: Dong-A University Research Foundation for Industry-Academy Cooperation, Busan (KR)

(21) Appl. No.: 12/774,104

(22) Filed: May 5, 2010

(30) Foreign Application Priority Data
Feb. 8, 2010 (KR) 10-2010-0011400

(51) Int. Cl. C12N 9/48

(52) U.S. Cl. 435/212

(57) ABSTRACT

Disclosed is serine protease isolated from Bombus ignitus, a bumble bee, capable of activating prothrombins and degrading fibrinogens and fibrins. Since the serine protease of the present invention enables to activate the prothrombin and directly degrade fibrinogens and fibrins it can be used in the development of a therapeutic agent for the treatment of thrombosis.

### CROSS-REFERENCE TO RELATED APPLICATION

This application claims under 35 U.S.C. §119(a) the benefit of Korean Patent Application No. 10-2010-0011400 filed Feb. 8, 2010, the entire contents of which are incorporated herein by reference.

### BACKGROUND

(a) Technical Field

The present disclosure relates to serine protease isolated from Bombus ignitus, a bumble bee, which is capable of activating prothrombins and directly degrading fibrinogens and fibrins.

(b) Background Art

Bees can protect their colonies from invaders such as other insects and animals by using venoms they carry in their body as a powerful defense means. Bee venoms comprise various kinds of venom proteins or peptides, for example, melittin [Gauldie et al., Eur. J. Biochem., 61:369-376 (1976)], phospholipase A₂ (PLA₂) [Six & Dennis, Biochim. Biophys. Acta 1488:1-19 (2000)], apamin [Banks et al., Nature 282:415-417 (1979)], hyaluronidase[Kreil, Protein Sci., 4:1666-1669 (1995)], serine protease [Winningham KM et al. J Allergy Clin Immunol 2004; 114:928-33], etc.

In oriental countries, there have been studies on various bee venom components to find the ways of their use in medicinal field [Mirshafiey A. Neuropharmacology 2007; 53:353-61]. In particular, honeybees and bumblebees which have been used as apiculture and pollen-mediating insects are more closely related to humans [Velthuis HHW et al. Apidologie 2006; 37:421-51].

In comparison, honey bees can release at least five times more of venom than the bumble bees, whereas the bumble bees can release venom a few times without losing stings [Hoffman DR et al. Ann Allergy 1984; 52:276-8]. Serine protease present in the bumble bee venom is one of the major components of the venom along with phospholipase A₂ (PLA₂) and bombolitin [Hoffman DR et al. J Allergy Clin Immunol 2001; 108:855-60].

Serine protease can be discovered in various living organisms and has a biochemical and structural property where amino acid residues including His, Asp, and Ser are conserved. Serine protease has versatile functions playing important roles in digestion, immune response, complement, cellular differentiation, and hemostasis [Neurath H. et al. Science 1984; 224:350-7; Krem MM. et al. Trends Biochem Sci 2002; 27:67-74]. In particular, the serine proteases present in snake venom, known as one of the major venoms, are known to be involved in hemostasis and thrombosis in mammals [Braud S et al. (2000) Biochimie 82:851-859; Matsui T et al. (2000) Biochim Biophys Acta 1477:146-156; Kini R M (2005) Pathophysiol Haemost Thrombo 34:200-204; Swenson S et al. (2005) Toxicon 45:1021-1039]. However, the gene of serine protease and its role in the mechanism of hemostasis and thrombosis has not been known.

### SUMMARY OF THE DISCLOSURE

The present invention has been completed by discovering that the serine protease contained in the venom of Bombus ignitus, a bumble bee, activates the prothrombin, and directly degrades fibrinogens and fibrins thus enabling to affect the blood coagulation mechanisms.

Therefore, in one aspect, the present invention provides serine protease derived from Bombus ignitus represented by SEQ. ID. NO. 1 capable of degrading fibrinogens and fibrins, one of major components involved in thrombosis.

In another aspect, the present invention provides a pharmaceutical composition for the treatment of thrombosis comprising serine protease isolated from the venom of Bombus ignitus.

### BRIEF DESCRIPTION OF THE DRAWINGS

The above and other features of the present invention will now be described in detail with reference to certain exemplary embodiments thereof illustrated the accompanying drawings which are given herein below by way of illustration only, and thus are not limitative of the present invention, and wherein:

FIG. 1 shows the nucleotide sequence of a cDNA of venom serine protease of Bombus ignitus, bumblebee, (Bi-VSP). The ‘ATG’ and ‘TAA’ codons in boxes represent the start codon and the stop codon, respectively.

FIG. 2 shows the deduced amino acid sequence of a cDNA of Bi-VSP. (*Open triangle (∇) divides between the free peptide as signal sequence and the propeptide including clip domain (clip domain has six strictly conserved cysteine residues, which form three pairs of disulfide bonds) while solid triangle (▾) divides between the propeptide including clip domain and serine protease domain.)

FIG. 3 shows the nucleotide sequence of a genomic DNA of Bi-VSP.

FIG. 4 shows the result of northern blot analysis of RNA extracted from fat body, midgut, muscle and venom gland of Bombus ignitus worker bee by using a cDNA of Bi-VSP as a probe.

FIG. 5 shows the result of purified recombinant Bi-proVSP. SDS-PAGE (left) and western blot (right) of the recombinant Bi-proVSP purified from baculovirus-infected insect cells. The anti-Bi-proVSP antibody was produced in mice injected with recombinant Bi-proVSP.

FIG. 6 shows the results that protein obtained from the venom gland, venom sac, and secreted venom of B. ignitus worker bees was analyzed using SDS-PAGE (left) and western blot (right). Bi-proVSP and Bi-VSP are shown. The arrowhead on the left indicates the position of Bi-proVSP. (*Bi-proVSP represents immature (i.e., inactivated) venom serine protease, and Bi-VSP represents mature (=activated) venom serine protease of Bombus ignitus).

FIG. 7 shows the result of glycoprotein staining of Bi-VSP purified from venom on SDS-PAGE. (*Horseradish peroxidase, which is a glycosylated protein, was used as a positive control. Soybean trypsin inhibitor, which is a non-glycosylated protein, was used as a negative control).

FIG. 8 shows the amino acid sequence alignment of Bi-VSP with known snake venom serine proteases. (* The residues in the conserved catalytic triad of the SP domain [His (H), Asp (D), and Ser (S)] are indicated using asterisks.)

FIG. 9 shows the result of SDS-PAGE analysis of human prothrombin activation by Bi-VSP. The number indicates the time (min) that prothrombin was incubated with Bi-VSP, according to time passage as described in Example 4.

FIG. 10 shows the result of SDS-PAGE analysis of human fibrinogen hydrolysis by Bi-VSP. The number indicates the time (min) that fibrinogen was incubated with Bi-VSP, according to time passage as described in Example 5.

FIG. 11 shows a picture for detection of the enzymatic activity of Bi-VSP on fibrin plates. Bi-VSP at various concentrations was dropped onto a fibrin plate and incubated for various periods of time as described in Example 6.

### DESCRIPTION OF DETAILED DISCLOSURE

The present disclosure relates to serine protease which is present in the venom of Bombus ignitus, a bumble bee, which is capable of activating prothrombins and degrading fibrinogens and fibrins.

The gene of serine protease present in the venom of Bombus ignitus was firstly isolated by the inventors of the present invention and it has not been well characterized yet. The inventors of the present invention filed a patent application in Korea on Feb. 17, 2009, regarding a nucleotide sequence for a gene encoding venom serine protease including the domain for serine protease, and assigned Korean Patent Application No. 10-2009-0013131. The domain of serine protease present in the venom of Bombus ignitus is a mature (i.e., activated) protein consisting of 247 amino acids as shown in SEQ. ID. NO. 1. This protein is much different from that in snake in terms of the size of amino acids and the sequence

The serine protease in the venom of Bombus ignitus can be obtained by extracting the venom stored in the venom sac, followed by gel filtration chromatography via Fast Protein Liquid Chromatography. The serine protease in the venom of Bombus ignitus enables to activate prothrombin, a blood coagulation factor, to thrombin, and also enables to degrade fibrinogens into fibrins, and subsequently into fibrin degradation products. Therefore, the serine protease of the present invention can be used for the treatment of deep vein thrombosis peripheral artery disease, and can reduce any medicinal accident which may occur after the angiosurgery, and also thrombosis which may recur.

The present invention is described in greater detail hereunder with reference to the examples but they should not be construed as limiting the scope of the present invention.

### EXAMPLES

### Example 1

### Cloning Genes for Serine Protease Present in the Venom of Bombus ignitus

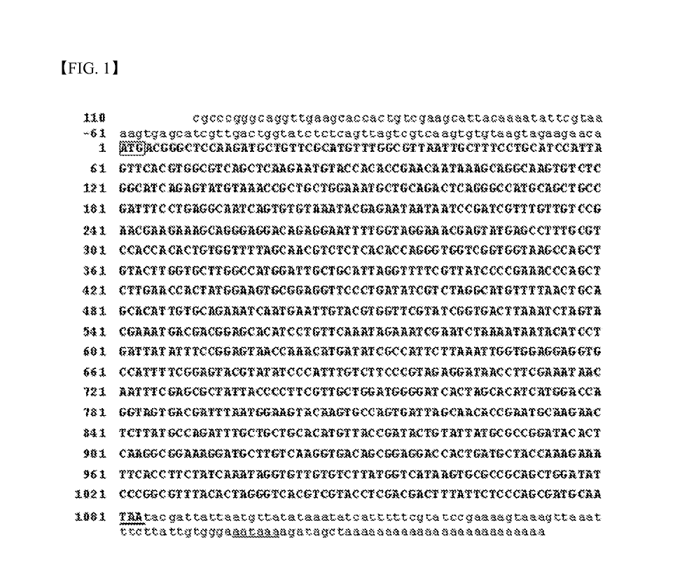

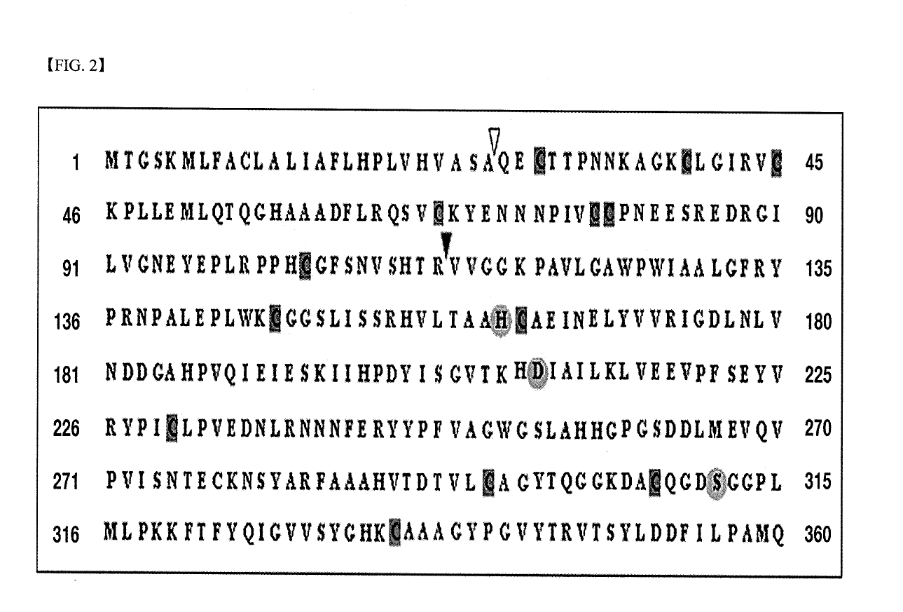

Total RNA was extracted from venom gland of Bombus ignitus worker bees provided by Dept. of Agricultural Biology in National Academy of Agricultural Science of Rural Development Administration, by using SV total RNA Isolation System kit (Promega, USA). Then, poly(A)+mRNA was extracted from the total RNA by using PolyATtract mRNA Isolation System kit (Promega, USA). Finally, a cDNA library was constructed by using the poly(A)+mRNA along with Uni-ZAP XR vector and Gigapack III Gold Packing Extract kit (Stratagene, USA), and expressed sequence tags (ESTs) were analyzed. DNA was extracted by using Wizard mini-preparation kit (Promega, USA) and its sequence was read by using automated DNA sequence analyzer (Applied Biosystems, USA). The nucleotide sequence was compared by using BLAST program of NCBI (http://www.ncbi.nlm.nih.gov/BLAST). As a result, a cDNA having the SEQ. ID. NO. 2 for the gene of the serine protease present in the venom of Bombus ignitus (Bi-VSP) was cloned (FIG. 1). The analysis of the SEQ. ID. NO. 3 (FIG. 2), an amino acid sequence deduced from the cDNA of the serine protease present in the venom of Bombus ignitus (Bi-VSP), revealed that it has a sequence homology with Holotrichia diomphalia PPAF-I (GenBank No. BAA34642), H. diomphalia PPAF-III (GenBank No. BAC15604), Bombyx mori PPAF-3 (GenBank No. AAL31707), Drosophila melanogaster MP1 (GenBank No. NP_{—}649560), D. melanogaster easter (GenBank No. NP_{—}524362) and Manduca sexta PAP-I (GenBank No. AAX18636), a group of PAP enzymes derived from insects, and also that cysteine (C) residues were conserved in clip domain while histidine (H), aspartic acid (D) and serine (S) residues were conserved in serine protease domain. Further, the above analysis also confirmed that the serine protease present in the venom of Bombus ignitus (Bi-VSP) comprises a prepeptide region consisting of 26 amino acids as a signal sequence, a propeptide region including a clip domain consisting of 87 amino acids, and a serine protease region consisting of 247 amino acids as a mature protein.

In addition, primers as shown in Table 1 below were prepared based on the serine protease present in the venom of Bombus ignitus (Bi-VSP), and genomic DNA of serine protease present in the venom of Bombus ignitus (Bi-VSP) was synthesized by PCR using the primers.

TABLE 1

| Primers | Nucleotide sequence | Location |
|---|---|---|
| Forward | 5′-ATG ACG GGC TCC | 1-24 |
| direction | AAG ATG CTG TTC-3′ | |
| primer 1 | (SEQ. ID. NO. 4) | |
| Reverse | 5′-TAC AGC TGG CTT | 363-340 |
| direction | ACC ACC GAC CAC-3′ | |
| primer 1 | (SEQ. ID. NO. 5) | |
| Forward | 5′-GTG GTC GGT GGT | 340-363 |
| direction | AAG CCA GCT GTA-3′ | |
| primer 2 | (SEQ. ID. NO. 6) | |
| Reverse | 5′-TTA TTG CAT CGC | 1083-1060 |
| direction | TGG GAG AAT AAA-3′ | |
| primer 2 | (SEQ. ID. NO. 7) | |

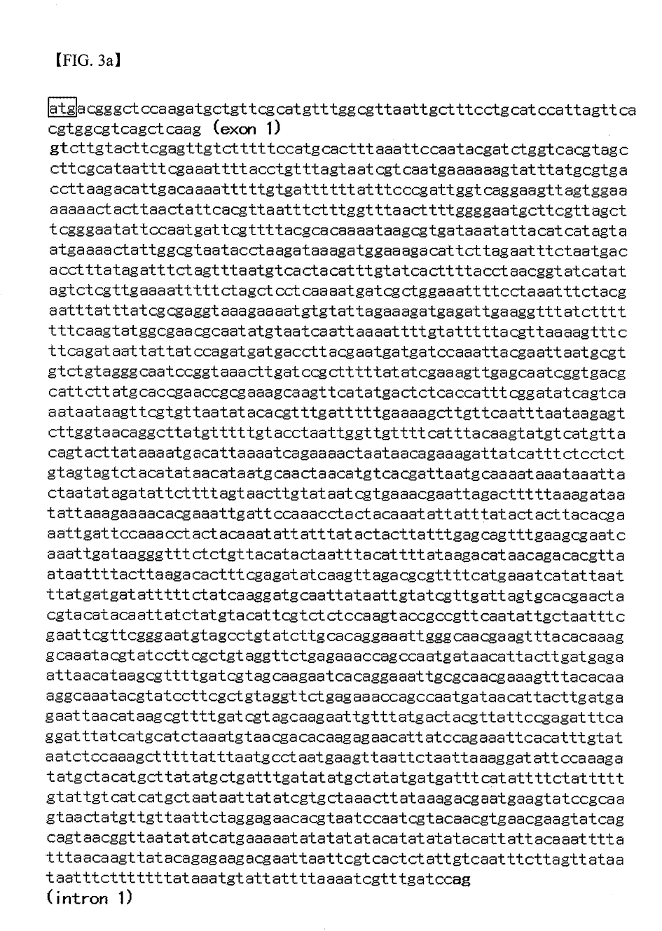

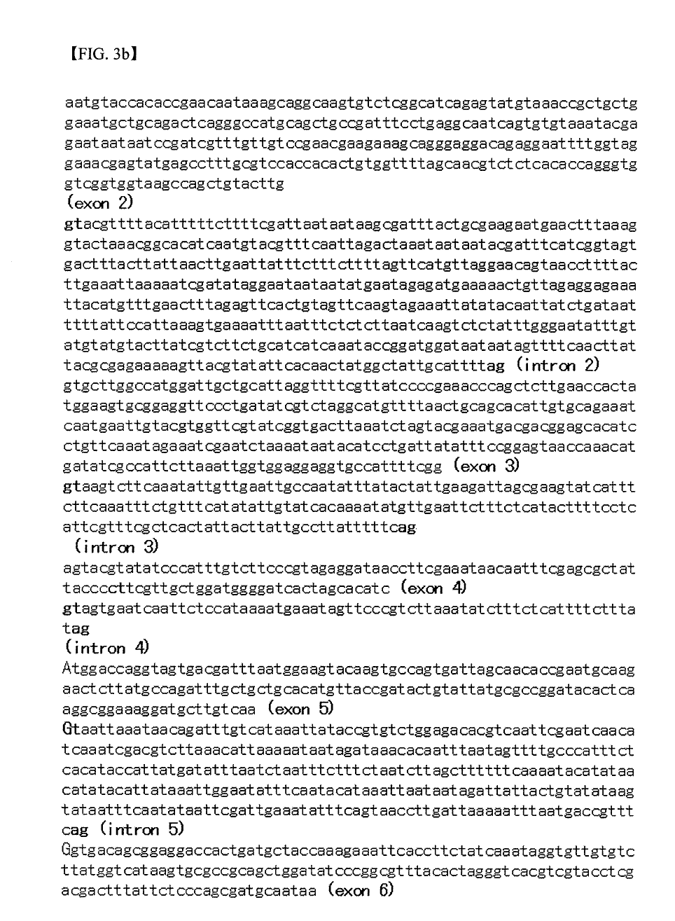

Genomic DNA of Bombus ignitus was isolated by using Wizard Genomic DNA Purification kit (Promega, USA), and then the genomic DNA containing the gene for the serine protease present in the venom of Bombus ignitus (Bi-VSP) was amplified by using the above primers and PCR premix kit (Bioneer Corp., Korea). The PCR reaction was conducted 35 cycles where each cycle was conducted under the condition of denaturation at 95° C. for 5 min, annealing at 60° C. for 1 min, and then polymerization at 720 for 3 min. Thus obtained amplified DNA was analyzed using an automated DNA sequence analyzer. The result revealed that the genomic DNA of the serine protease present in the venom of Bombus ignitus (BI-VSP) consists of 6 exons and 5 introns, where the entire length of the above genomic DNA from the start codon to termination codon is 4505 bp long (FIG. 3).

### Example 2

### Venom Gland-Specific Expression, Cleavage and O-Glycosylation of Serine Protease Present in the Venom of Bombus ignitus (Bi-VSP)

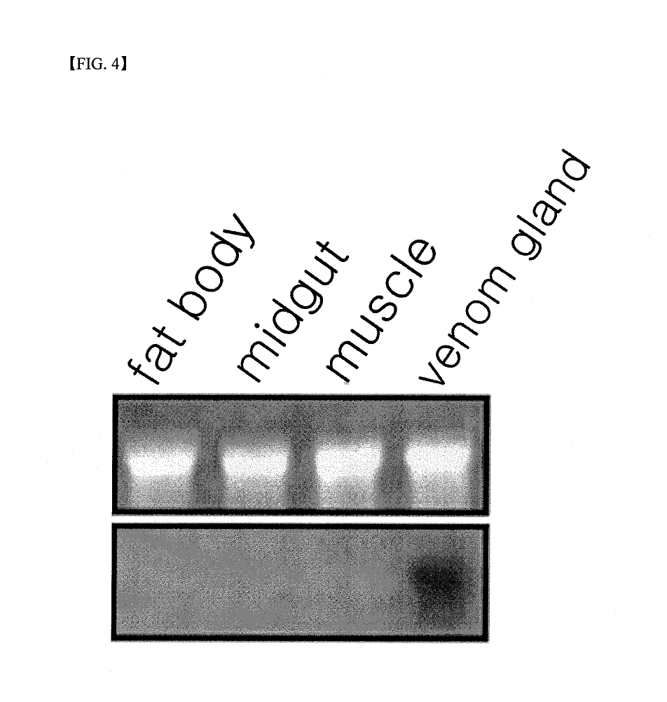

RNA was extracted from fat body, midgut, muscle and venom gland of Bombus ignitus by using a Total RNA isolation kit (Promega, USA). Thus obtained RNA was electrophoresed in a 1.0% formaldehyde agarose gel after loading 5 μg per each well, the gel was transferred onto a nylon blotting membrane (Schleicher & Schuell, Germany), and then hybridized at 42° C. with a probe of [a-³²P]dCTP (A mersham, USA)-labelled cDNA of serine protease present in the venom of Bombus ignitus (Bi-VSP). As a result, mRNA of the serine protease present in the venom of Bombus ignitus (Bi-VSP) was discovered in a venom gland-specific pattern (FIG. 4).

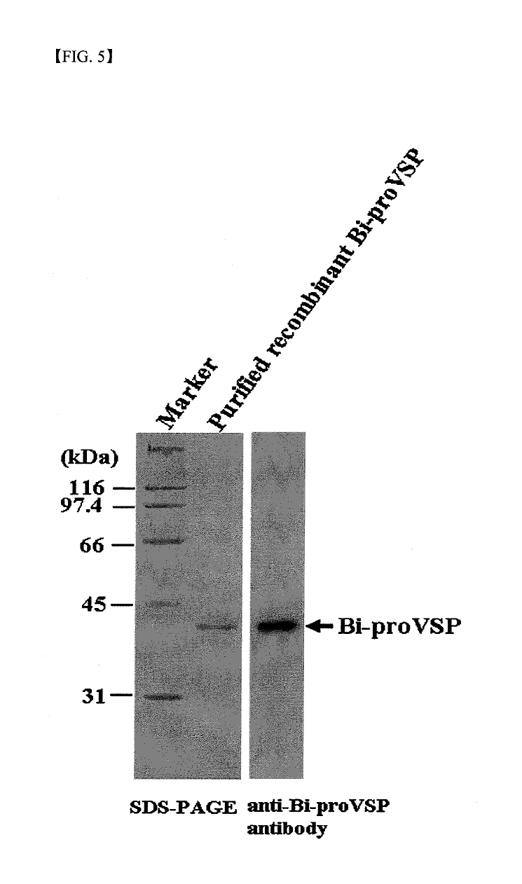

In order to prepare antibodies against the serine protease present in the venom of Bombus ignitus (Bi-VSP), the cDNA of the serine protease present in the venom of Bombus ignitus (Bi-VSP) was inserted into a BamH I-Xho I region of an insect Autographa californica nucleopolyhedrovirus transfection vector pBAC1 (Clontech, USA), and then co-transfected to an insect cell line Sf9 (Spodoptera frugiperda 9) along with 100 ng of the transfection vector and 500 ng of bAcGOZA viral DNA [Je et al., Biotechnol. Lett., 23:575-582 (2001)] by using Lipofectin (Clonetech, USA). Five days later, the resulting culture was collected and a recombinant Autographa californica nucleopolyhedrovirus which expresses the recombinant venom serine protease of Bombus ignitus (Bi-proVSP) was prepared. The recombinant Autographa californica nucleopolyhedrovirus was grown in Sf9 cell line, and the recombinant venom serine protease (Bi-proVSP) was separated by using a HisTrap column (Amersham Bioscience, USA). The separated recombinant venom serine protease (Bi-proVSP) was injected into Balb/c mice to produce polyclonal antibodies [Choo et al., Mol. Cell. Neurisci., 38:224-235 (2008)]. Western blot was performed by using the separated serine protease present in the venom of Bombus ignitus (Bi-VSP) and the above antibodies [FIG. 5].

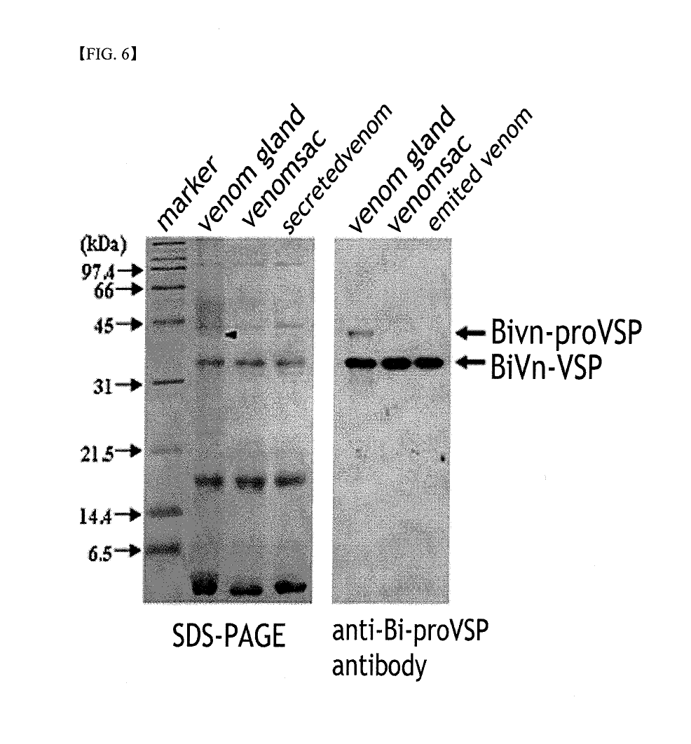

Venom protein samples were obtained from venom gland, venom sac, emitted venom, and they were electrophoresed in a 15% SDS-PAGE gel, and then Western blot was performed by using the above antibodies. As a result, it was found that both an inactivated form of venom serine protease present in the venom of Bombus ignitus (Bi-proVSP) and an activated form of venom serine protease present in the venom of Bombus ignitus (Bi-VSP) were observed in venom gland, whereas only an activated form of venom serine protease present in the venom of Bombus ignitus (Bi-VSP) was observed in venom sac and emitted venom [FIG. 6]. Therefore, it was confirmed that the serine protease present in the venom of Bombus ignitus (Bi-VSP) is expressed in venom gland, cleaved in an activated form, stored in a venom sac, and then emitted.

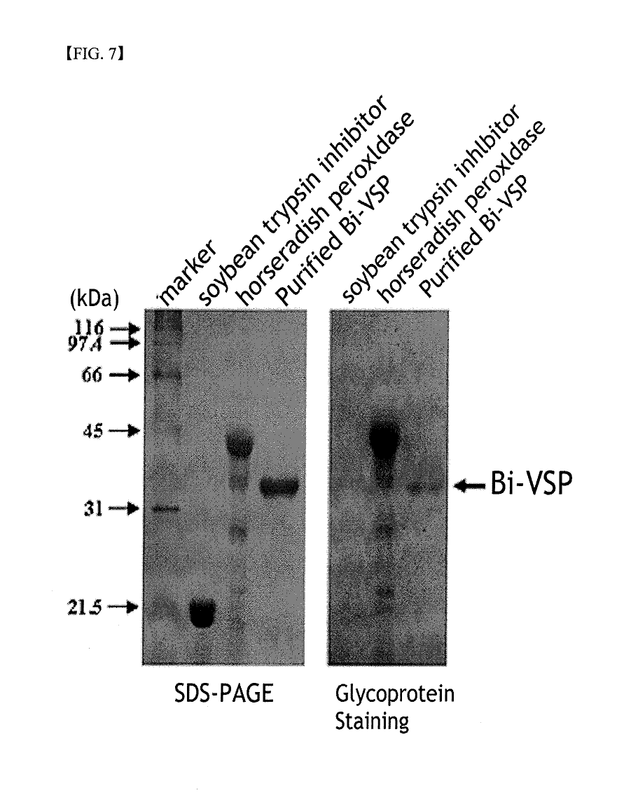

In order to examine the region where the serine protease present in the venom of Bombus ignitus (Bi-proVSP) is cleaved in the activated form of venom serine protease (Bi-VSP), the serine protease present in the venom of Bombus ignitus (Bi-VSP) with 34 kDa was transferred onto a polyvinylidene difluoride, PVDF membrane (Applied Biosystems, USA) and then analyzed the N-terminal region via Edman degradation method. As a result, it was confirmed that, as shown in FIG. 2, the serine protease present in the venom of Bombus ignitus (Bi-proVSP) is cleaved between the 113^{th} amino acid, Arg, and the 114^{th} amino acid, Val, thereby being converted into an activated form of venom serine protease (Bi-VSP), comprising serine protease which consists of 247 amino acids. That is, the serine protease of the present invention is an activated form of venom serine protease which consists of 247 amino acids as represented by SEQ. ID. NO. 1. The estimated molecular weight of the serine protease consisting of 247 amino acids by calculation is 27 kDa. However, it appears to have 34 kDa on a SDS-PAGE gel because it contains about 20% of sugar. The serine protease in the venom of Bombus ignitus did not have a N-glycosylation domain but had a O-glycosylation domain. To confirm this, the serine protease in the venom of Bombus ignitus in an activated form was subject to glycoprotein staining by using Gel/Code glycoprotein staining kit (Pierce, USA), and as a result, it was confirmed that the serine protease in the venom of Bombus ignitus in an activated form is a glycoprotein with O-glycosylation (FIG. 7).

### Example 3

### Comparison of Amino Acids Sequences Between Serine Proteases Present in the Venom of Bombus ignitus and that in Snake

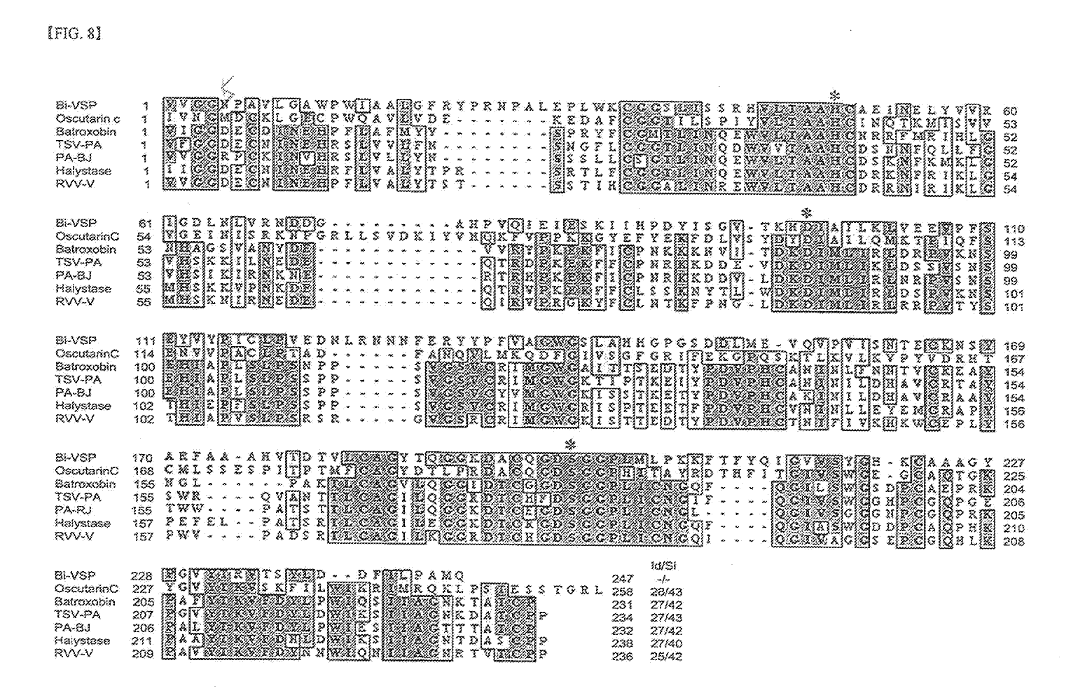

The nucleotide sequences of the serine protease in the venom of Bombus ignitus and that in snake were compared by using the BLAST program of NCBI (http://www.ncbi.nlm.nih.gov/BLAST). When the amino acids sequences of the above two serine proteases were compared, it was found that serine protease of bombus ignitus (Bi-VSP) had a certain extent of homology with Oscutarin C which serves as a prothrombin activator in blood coagulation mechanism (GenBank No. AY940204); Batroxobin which has similar activity as thrombin (GenBank No. AAA48553); TSV-PA which activates plasmin precursor (GenBank No. Q91516); PA-BJ (GenBank No. P81824); Halystase (GenBank No. P81176) and RVV-V (GenBank No. P18964), and histidine, aspartic acid, and serine residues were well conserved in the serine protease domain (FIG. 8).

### Example 4

### Role of the Serine Protease Present in Venom of Bombus ignitus as a Prothrombin Activator

In order to examine the function of venom serine protease of Bombus ignitus (Bi-VSP) whether it activates prothrombin, a precursor of thrombin which plays a crucial role in blood coagulation, the following experiment was performed.

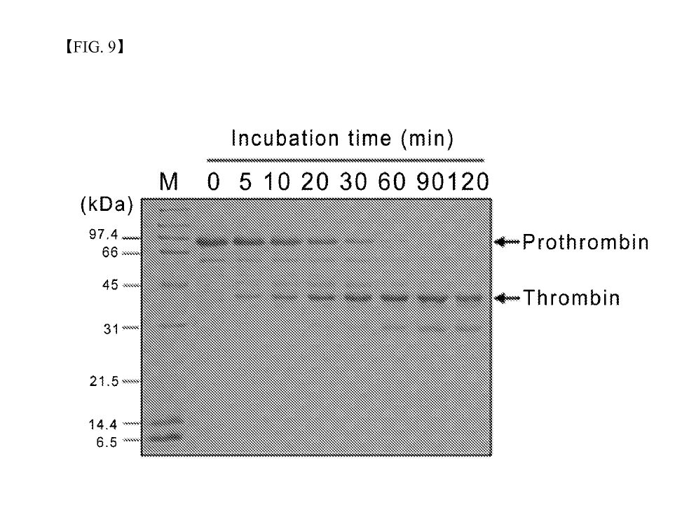

2 μg of prothrombin (Sigma), a human blood coagulation factor, and 2 ng of purified serine protease from the venom sac of Bombus ignitus were diluted in 50 mM Tris-HCl (pH 8.0) buffer containing 100 mM NaCl and M CaCl₂, and reacted at 37□ and then the mixture was run in a 14% SDS-PAGE gel and observed the result according to time passage [Speijer H et al. J Biol Chem 1986; 261:13258-67]. As a result, it was found that prothrombin started to convert into an activated form of thrombin 5 min after the reaction and was completely converted to thrombin 60 min after the reaction (FIG. 9). This was similar to the mechanism of factor Xa, a blood coagulation factor, in blood coagulation mechanism.

### Example 5

### Role of the Serine Protease in the Venom of Bombus ignitus as an Enzyme for Fibrinogenolysis

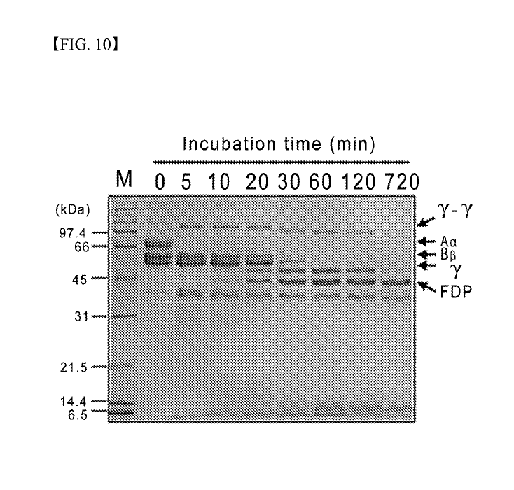

10 μg of fibrinogen (MP Biomedicals, Solon, Ohio, USA), a human fibrin precursor, and 0.25 μg of purified serine protease from the venom sac of Bombus ignitus were diluted in 50 mM Tris-HCl (pH 8.0) buffer and reacted at 37° C. and then the mixture was run in a 14% SDS-PAGE gel and observed the result according to time passage [Matsui T et al. Eur J Biochem 1998; 252:569-75]. As a result, it was found that the serine protease in the venom of Bombus ignitus (Bi-VSP) did not show any fibrin clot but hydrolyzed the chains of fibrinogen Aα, Bβ, γ. Aα chain was completely hydrolyzed within 5 min after the reaction, while Bβ and γ chains were completely hydrolyzed within 60 min. This suggests that the serine protease in the venom of Bombus ignitus (Bi-VSP) has the thrombin-like activity capable of hydrolyzing fibrinogen. Further, it was found that between 60 min and 720 min all fibrins, which were from fibrinogens, were completely converted into fibrin degradation products (FDP). This further confirms that the serine protease in the venom of Bombus ignitus (Bi-VSP) has the plasmin-like activity (capable of degrading fibrin (FIG. 10).

### Example 6

### Assay on the Role of the Serine Protease in the Venom of Bombus ignitus as an enzyme for fibrinolysis

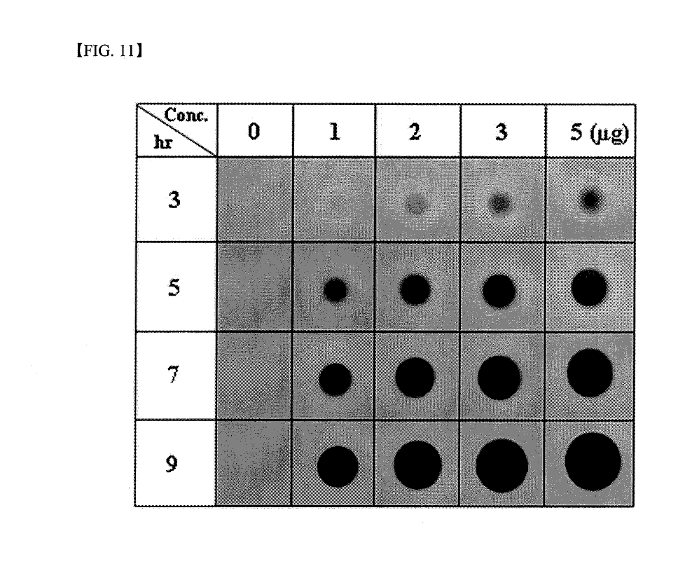

As shown in Example 5, through the experiment of fibrinogen reaction with the serine protease in the venom of Bombus ignitus (Bi-VSP), it was confirmed that the serine protease in the venom of Bombus ignitus (Bi-VSP) not only specifically lyses fibrinogens into fibrins but also converts the fibrins into fibrin degradation products on a SDS-PAGE gel. In addition, fibrin plate assay was conducted in order to obtain a more specific and persuasive result on the fibrinolytic activity of the serine protease in the venom of Bombus ignitus. Fibrinogens (0.6%/10 mL) was added into a Borate buffer (pH 7.8), lysed for 1 hr at 30° C. Then, 10 mL of the resultant was transferred into a plate to convert the fibrinogens into fibrins, and 40 units of thrombin were added thereto to dilute and allowed to react at room temperature to make it solid[Astrup T. et al. Arch. Biochem. Biophys. (1991). 40, 346-351]. To the fibrin plate was added the purified serine protease in the venom of Bombus ignitus in varying concentrations (0, 1, 2, 3, and 5 tag) and allowed to react at 37° C. for a period of 3, 5, 7, 9 hrs, respectively, and observed the fibrinolytic activity. As a result, it was found that white zones for the fibirnolysis were formed according to each different concentration (FIG. 11), and thus confirmed that the serine protease in the venom of Bombus ignitus is capable of effective fibrinolysis.

### ADVANTAGEOUS EFFECTS

The serine protease of the present invention enables to activate the prothrombin and directly degrade fibrinogens and fibrins and thus it can be used in the development of a therapeutic agent for the treatment of thrombosis.

It will be clear to one of skill in the art that the present invention may be embodied in other forms, structures, arrangements, and proportions, and may use other elements, materials and components. The present disclosed embodiments are, therefore, to be considered in all respects as illustrative and not restrictive, the scope of the invention being indicated by the appended claims and not limited to the foregoing description.

1. Serine protease isolated from the venom of Bombus ignitus represented by SEQ. ID. NO. 1 capable of activating prothrombin.

2. Serine protease isolated from the venom of Bombus ignitus represented by SEQ. ID. NO. 1 capable of degrading fibrinogen into fibrin.

3. Serine protease isolated from the venom of Bombus ignitus represented by SEQ. ID. NO. 1 capable of degrading fibrin.

4. A pharmaceutical composition for the treatment of thrombosis comprising serine protease isolated from the venom of Bombus ignitus according to claim 1.

5. A pharmaceutical composition for the treatment of thrombosis comprising serine protease isolated from the venom of Bombus ignitus according to claim 2.

6. A pharmaceutical composition for the treatment of thrombosis comprising serine protease isolated from the venom of Bombus ignitus according to claim 3.


SEQUENCE LISTING (15 sequences)

SEQ ID NO: 1 - protein, 247 aa, Bombus ignitus
VVGGKPAVLGAWPWIAALGFRYPRNPALEPLWKCGGSLISSRHVLTAAHCAEINELYVVRIGDLNLVRNDDGAHPVQIEIESKIIHPDYISGVTKHDIAILKLVEEVPFSEYVYPICLPVEDNLRNNNFERYYPFVAGWGSLAHHGPGSDDLMEVQVPVISNTECKNSYARFAAAHVTDTVLCAGYTQGGKDACQGDSGGPLMLPKKFTFYQIGVVSYGHKCAAAGYPGVYTRVTSYLDDFILPAMQ

SEQ ID NO: 2 - DNA, 1083 bp, Bombus ignitus
atgacgggctccaagatgctgttcgcatgtttggcgttaattgctttcctgcatccattagttcacgtggcgtcagctcaagaatgtaccacaccgaacaataaagcaggcaagtgtctcggcatcagagtatgtaaaccgctgctggaaatgctgcagactcagggccatgcagctgccgatttcctgaggcaatcagtgtgtaaatacgagaataataatccgatcgtttgttgtccgaacgaagaaagcagggaggacagaggaattttggtaggaaacgagtatgagcctttgcgtccaccacactgtggttttagcaacgtctctcacaccagggtggtcggtggtaagccagctgtacttggtgcttggccatggattgctgcattaggttttcgttatccccgaaacccagctcttgaaccactatggaagtgcggaggttccctgatatcgtctaggcatgttttaactgcagcacattgtgcagaaatcaatgaattgtacgtggttcgtatcggtgacttaaatctagtacgaaatgacgacggagcacatcctgttcaaatagaaatcgaatctaaaataatacatcctgattatatttccggagtaaccaaacatgatatcgccattcttaaattggtggaggaggtgccattttcggagtacgtatatcccatttgtcttcccgtagaggataaccttcgaaataacaatttcgagcgctattaccccttcgttgctggatggggatcactagcacatcatggaccaggtagtgacgatttaatggaagtacaagtgccagtgattagcaacaccgaatgcaagaactcttatgccagatttgctgctgcacatgttaccgatactgtattatgcgccggatacactcaaggcggaaaggatgcttgtcaaggtgacagcggaggaccactgatgctaccaaagaaattcaccttctatcaaataggtgttgtgtcttatggtcataagtgcgccgcagctggatatcccggcgtttacactagggtcacgtcgtacctcgacgactttattctcccagcgatgcaataa

SEQ ID NO: 3 - protein, 360 aa, Bombus ignitus
MTGSKMLFACLALIAFLHPLVHVASAQECTTPNNKAGKCLGIRVCKPLLEMLQTQGHAAADFLRQSVCKYENNNPIVCCPNEESREDRGILVGNEYEPLRPPHCGFSNVSHTRVVGGKPAVLGAWPWIAALGFRYPRNPALEPLWKCGGSLISSRHVLTAAHCAEINELYVVRIGDLNLVRNDDGAHPVQIEIESKIIHPDYISGVTKHDIAILKLVEEVPFSEYVYPICLPVEDNLRNNNFERYYPFVAGWGSLAHHGPGSDDLMEVQVPVISNTECKNSYARFAAAHVTDTVLCAGYTQGGKDACQGDSGGPLMLPKKFTFYQIGVVSYGHKCAAAGYPGVYTRVTSYLDDFILPAMQ

SEQ ID NO: 4 - DNA, 24 bp, Artificial Sequence
  Description of Artificial Sequence Synthetic PAP forward primer 1
atgacgggctccaagatgctgttc

SEQ ID NO: 5 - DNA, 24 bp, Artificial Sequence
  Description of Artificial Sequence Synthetic PAP reverse primer 1
tacagctggcttaccaccgaccac

SEQ ID NO: 6 - DNA, 24 bp, Artificial Sequence
  Description of Artificial Sequence Synthetic PAP forward primer 2
gtggtcggtggtaagccagctgta

SEQ ID NO: 7 - DNA, 24 bp, Artificial Sequence
  Description of Artificial Sequence Synthetic PAP reverse primer 2
ttattgcatcgctgggagaataaa

SEQ ID NO: 8 - DNA, 1304 bp, Bombus ignitus
cgcccgggcaggttgaagcaccactgtcgaagcattacaaaatattcgtaaaagtgagcatcgttgactggtatctctcagttagtcgtcaagtgtgtaagtagaagaacaatgacgggctccaagatgctgttcgcatgtttggcgttaattgctttcctgcatccattagttcacgtggcgtcagctcaagaatgtaccacaccgaacaataaagcaggcaagtgtctcggcatcagagtatgtaaaccgctgctggaaatgctgcagactcagggccatgcagctgccgatttcctgaggcaatcagtgtgtaaatacgagaataataatccgatcgtttgttgtccgaacgaagaaagcagggaggacagaggaattttggtaggaaacgagtatgagcctttgcgtccaccacactgtggttttagcaacgtctctcacaccagggtggtcggtggtaagccagctgtacttggtgcttggccatggattgctgcattaggttttcgttatccccgaaacccagctcttgaaccactatggaagtgcggaggttccctgatatcgtctaggcatgttttaactgcagcacattgtgcagaaatcaatgaattgtacgtggttcgtatcggtgacttaaatctagtacgaaatgacgacggagcacatcctgttcaaatagaaatcgaatctaaaataatacatcctgattatatttccggagtaaccaaacatgatatcgccattcttaaattggtggaggaggtgccattttcggagtacgtatatcccatttgtcttcccgtagaggataaccttcgaaataacaatttcgagcgctattaccccttcgttgctggatggggatcactagcacatcatggaccaggtagtgacgatttaatggaagtacaagtgccagtgattagcaacaccgaatgcaagaactcttatgccagatttgctgctgcacatgttaccgatactgtattatgcgccggatacactcaaggcggaaaggatgcttgtcaaggtgacagcggaggaccactgatgctaccaaagaaattcaccttctatcaaataggtgttgtgtcttatggtcataagtgcgccgcagctggatatcccggcgtttacactagggtcacgtcgtacctcgacgactttattctcccagcgatgcaataatacgattattaatgttatataaatatcatttttcgtatccgaaaagtaaagttaaatttcttattgtgggaaataaaagatagctaaaaaaaaaaaaaaaaaaaaaaaaa

SEQ ID NO: 9 - DNA, 4505 bp, Bombus ignitus
atgacgggctccaagatgctgttcgcatgtttggcgttaattgctttcctgcatccattagttcacgtggcgtcagctcaaggtcttgtacttcgagttgtctttttccatgcactttaaattccaatacgatctggtcacgtagccttcgcataatttcgaaattttacctgtttagtaatcgtcaatgaaaaaagtatttatgcgtgaccttaagacattgacaaaatttttgtgattttttatttcccgattggtcaggaagttagtggaaaaaaactacttaactattcacgttaatttctttggtttaacttttggggaatgcttcgttagcttcgggaatattccaatgattcgttttacgcacaaaataagcgtgataaatattacatcatagtaatgaaaactattggcgtaatacctaagataaagatggaaagacattcttagaatttctaatgacacctttatagatttctagtttaatgtcactacatttgtatcacttttacctaacggtatcatatagtctcgttgaaaatttttctagctcctcaaaatgatcgctggaaattttcctaaatttctacgaatttatttatcgcgaggtaaagaaaatgtgtattagaaagatgagattgaaggtttatctttttttcaagtatggcgaacgcaatatgtaatcaattaaaattttgtatttttacgttaaaagtttcttcagataattattatccagatgatgaccttacgaatgatgatccaaattacgaattaatgcgtgtctgtagggcaatccggtaaacttgatccgctttttatatcgaaagttgagcaatcggtgacgcattcttatgcaccgaaccgcgaaagcaagttcatatgactctcaccatttcggatatcagtcaaataataagttcgtgttaatatacacgtttgatttttgaaaagcttgttcaatttaataagagtcttggtaacaggcttatgtttttgtacctaattggttgttttcatttacaagtatgtcatgttacagtacttataaaatgacattaaaatcagaaaactaataacagaaagattatcatttctcctctgtagtagtctacatataacataatgcaactaacatgtcacgattaatgcaaaataaataaattactaatatagatattcttttagtaacttgtataatcgtgaaacgaattagactttttaaagataatattaaagaaaacacgaaattgattccaaacctactacaaatattatttatactacttacacgaaattgattccaaacctactacaaatattatttatactacttatttgagcagtttgaagcgaatcaaattgataagggtttctctgttacatactaatttacattttataagacataacagacacgttaataattttacttaagacactttcgagatatcaagttagacgcgttttcatgaaatcatattaatttatgatgatatttttctatcaaggatgcaattataattgtatcgttgattagtgcacgaactacgtacatacaattatctatgtacattcgtctctccaagtaccgccgttcaatattgctaatttcgaattcgttcgggaatgtagcctgtatcttgcacaggaaattgggcaacgaagtttacacaaaggcaaatacgtatccttcgctgtaggttctgagaaaccagccaatgataacattacttgatgagaattaacataagcgttttgatcgtagcaagaatcacaggaaattgcgcaacgaaagtttacacaaaggcaaatacgtatccttcgctgtaggttctgagaaaccagccaatgataacattacttgatgagaattaacataagcgttttgatcgtagcaagaattgtttatgactacgttattccgagatttcaggatttatcatgcatctaaatgtaacgacacaagagaacattatccagaaattcacatttgtataatctccaaagctttttatttaatgcctaatgaagttaattctaattaaaggatattccaaagatatgctacatgcttatatgctgatttgatatatgctatatgatgatttcatattttctatttttgtattgtcatcatgctaataattatatcgtgctaaacttataaagacgaatgaagtatccgcaagtaactatgttgttaattctaggagaacacgtaatccaatcgtacaacgtgaacgaagtatcagcagtaacggttaatatatcatgaaaaatatatatatacatatatatacattattacaaattttatttaacaagttatacagagaagacgaattaattcgtcactctattgtcaatttcttagttataataatttctttttttataaatgtattattttaaaatcgtttgatccagaatgtaccacaccgaacaataaagcaggcaagtgtctcggcatcagagtatgtaaaccgctgctggaaatgctgcagactcagggccatgcagctgccgatttcctgaggcaatcagtgtgtaaatacgagaataataatccgatcgtttgttgtccgaacgaagaaagcagggaggacagaggaattttggtaggaaacgagtatgagcctttgcgtccaccacactgtggttttagcaacgtctctcacaccagggtggtcggtggtaagccagctgtacttggtacgttttacatttttcttttcgattaataataagcgatttactgcgaagaatgaactttaaaggtactaaacggcacatcaatgtacgtttcaattagactaaataataatacgatttcatcggtagtgactttacttattaacttgaattatttctttcttttagttcatgttaggaacagtaaccttttacttgaaattaaaaatcgatataggaataataatatgaatagagatgaaaaactgttagaggagaaattacatgtttgaactttagagttcactgtagttcaagtagaaattatatacaattatctgataatttttattccattaaagtgaaaatttaatttctctcttaatcaagtctctatttgggaatatttgtatgtatgtacttatcgtcttctgcatcatcaaataccggatggataataatagttttcaacttattacgcgagaaaaagttacgtatattcacaactatggctattgcattttaggtgcttggccatggattgctgcattaggttttcgttatccccgaaacccagctcttgaaccactatggaagtgcggaggttccctgatatcgtctaggcatgttttaactgcagcacattgtgcagaaatcaatgaattgtacgtggttcgtatcggtgacttaaatctagtacgaaatgacgacggagcacatcctgttcaaatagaaatcgaatctaaaataatacatcctgattatatttccggagtaaccaaacatgatatcgccattcttaaattggtggaggaggtgccattttcgggtaagtcttcaaatattgttgaattgccaatatttatactattgaagattagcgaagtatcatttcttcaaatttctgtttcatatattgtatcacaaaatatgttgaattctttctcatacttttcctcattcgtttcgctcactattacttattgccttatttttcagagtacgtatatcccatttgtcttcccgtagaggataaccttcgaaataacaatttcgagcgctattaccccttcgttgctggatggggatcactagcacatcgtagtgaatcaattctccataaaatgaaatagttcccgtcttaaatatctttctcattttctttatagatggaccaggtagtgacgatttaatggaagtacaagtgccagtgattagcaacaccgaatgcaagaactcttatgccagatttgctgctgcacatgttaccgatactgtattatgcgccggatacactcaaggcggaaaggatgcttgtcaagtaattaaataacagatttgtcataaattataccgtgtctggagacacgtcaattcgaatcaacatcaaatcgacgtcttaaacattaaaaataatagataaacacaatttaatagttttgcccatttctcacataccattatgatatttaatctaatttctttctaatcttagcttttttcaaaatacatataacatatacattataaattggaatatttcaatacataaattaataatagattattactgtatataagtataatttcaatataattcgattgaaatatttcagtaaccttgattaaaaatttaatgaccgtttcagggtgacagcggaggaccactgatgctaccaaagaaattcaccttctatcaaataggtgttgtgtcttatggtcataagtgcgccgcagctggatatcccggcgtttacactagggtcacgtcgtacctcgacgactttattctcccagcgatgcaataa

SEQ ID NO: 10 - protein, 258 aa, Oxyuranus scutellatus
IVNGMDCKLGECPWQAVLVDEKEDAFCGGTILSPIYVLTAAHCINQTKMISVVVGEINISRKNPGRLLSVDKIYVHQKFVPPKKGYEFYEKFDLVSYDYDIAILQMKTPIQFSENVVPACLPTADFANQVLMKQDFGIVSGFGRIFEKGPQSKTLKVLKVPYVDRHTCMLSSESPITPTMFCAGYDTLPRDACQGDSGGPHITAYRDTHFITGIVSWGEGCAQTGKYGVYTKVSKFILWIKRIMRQKLPSTESSTGRL

SEQ ID NO: 11 - protein, 231 aa, Bothrops atrox
VIGGDECDINEHPFLAFMYYSPRYFCGMTLINQEWVLTAAHCNRRFMRIHLGNHAGSVANYDEVVRYPKEKFICPNKKKNVITDKDIMLIRLDRPVKNSEHIAPLSLPSNPPSVGSVCRIMGWGAITTSEDTYPDVPHCANINLFNNTVCREAYNGLPAKTLCAGVLQGGIDTCGGDSGGPLICNGQFQGILSWGSDPCAEPRKPAFYTKVFDYLPWIQSIIAGNKTATCP

SEQ ID NO: 12 - protein, 234 aa, Viridovipera stejnegeri
VFGGDECNINEHRSLVVLFNSNGFLCGGTLINQDWVVTAAHCDSNNFQLLFGVHSKKILNEDEQTRDPKEKFFCPNRKKDDEVDKDIMLIKLDSSVSNSEHIAPLSLPSSPPSVGSVCRIMGWGKTIPTKEIYPDVPHCANINILDHAVCRTAYSWRQVANTTLCAGILQGGRDTCHFDSGGPLICNGIFQGIVSWGGHPCGQPGEPGVYTKVFDYLDWIKSIIAGNKDATCPP

SEQ ID NO: 13 - protein, 232 aa, Bothrops jararaca
VVGGRPCKINVHRSLVLLYNSSSLLCSGTLINQEWVLTAAHCDSKNFKMKLGVHSIKIRNKNERTRHPKEKFICPNRKKDDVLDKDIMLIRLNRPVSNSEHIAPLSLPSSPPSVGSVCYVMGWGKISSTKETYPDVPHCAKINILDHAVCRAAYTWWPATSTTLCAGILQGGKDTCEGDSGGPLICNGLQGIVSGGGNPCGQPRKPALYTKVFDYLPWIESIIAGTTTATCP

SEQ ID NO: 14 - protein, 238 aa, Gloydius blomhoffi
IIGGDECNINEHRFLVALYTPRSRTLFCGGTLINQEWVLTAAHCDRKNFRIKLGMHSKKVPNKDEQTRVPKEKFFCLSSKNYTLWDKDIMLIRLDSPVKNSTHIEPFSLPSSPPSVGSVCRIMGWGRISPTEETFPDVPHCVNINLLEYEMCRAPYPEFELPATSRTLCAGILEGGKDTCRGDSGGPLICNGQFQGIASWGDDPCAQPHKPAAYTKVFDHLDWIKSIIAGNTDASCPP

SEQ ID NO: 15 - protein, 236 aa, Daboia russellii
VVGGDECNINEHPFLVALYTSTSSTIHCGGALINREWVLTAAHCDRRNIRIKLGMHSKNIRNEDEQIRVPRGKYFCLNTKFPNGLDKDIMLIRLRRPVTYSTHIAPVSLPSRSRGVGSRCRIMGWGKISTTEDTYPDVPHCTNIFIVKHKWCEPLYPWVPADSRTLCAGILKGGRDTCHGDSGGPLICNGQIQGIVAGGSEPCGQHLKPAVYTKVFDYNNWIQNIIAGNRTVTCPP